(19) United States
(12) Patent Application Publication
(10) Pub. No.: US 2023/0165447 A1
(43) Pub. Date: Jun. 1, 2023

(54) EXTERNAL SLEEVE PROVIDING ADDITIONAL WORKING CHANNEL

(71) Applicant: Boston Scientific Scimed, Inc., Maple Grove (MN)

(72) Inventors: Richard Crawford, Athenry (IE); Enda Connaughton, Galway (IE)

(73) Assignee: Boston Scientific Scimed, Inc., Maple Grove, MN (US)

(21) Appl. No.: 18/071,211

(22) Filed: Nov. 29, 2022

Related U.S. Application Data
(60) Provisional application No. 63/284,269, filed on Nov. 30, 2021.

(51) Int. Cl. A61B 1/018; A61B 1/00

(52) U.S. Cl. CPC A61B 1/018; A61B 1/0008

(57) ABSTRACT

A sheath system configured to be operatively engaged with a delivery device with a working channel through which a medical instrument is passed. The sheath system thus increases the number of working channels of the flexible tubular system to permit more than one medical instrument to be navigated to and used at a treatment site. The sheath system has a low profile during delivery, yet may be shifted into a configuration facilitating passage of medical instruments therethrough.

### CROSS-REFERENCE To RELATED APPLICATIONS

This application claims the benefit of priority of U.S. Provisional Application No. 63/284,269, filed Nov. 30, 2021, the entire disclosure of which is hereby incorporated by reference herein for all purposes.

### FIELD

The present disclosure relates generally to the field of devices, such as endoscopes and the like, with interior working channels. More particularly, the present disclosure relates to devices, systems, and methods for providing an additional working channel, such as an external working channel, to a device with limited internal working channels.

### BACKGROUND

Various tubular flexible medical devices, such as endoscopes, exist for insertion into a body passageway or cavity to enable a medical professional to perform surgical procedures at a treatment site internal to the patient's body. In general, an endoscope includes a long flexible tubular member equipped with, for example, a miniature viewing device (optical component, such as a camera), an illumination device (e.g., an LED or optical fiber), and working channels. The endoscope has a proximal end that remains external to the patient, and a distal end having an endoscope tip configured for insertion into the patient. The distal end is navigated within the patient (through one or more body passages) and is delivered to an internal treatment site. Such tubular devices as endoscopes generally have a limited number of working channels, and the outer diameter of such devices generally is limited by the size of the body passage through which the device is to be navigated.

There remains a need for increasing the number of working channels of a delivery device (such as an endoscope) which is navigated through the body and which provides a passage for surgical instruments to be delivered to a treatment site, while optimizing the mobility of the surgical instruments to be delivered to and used at the treatment site, while nonetheless maintaining the required dimension constraints to permit travel of the delivery device through body passages of the patient.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an additional external working channel is provided with respect to a delivery device with at least one working channel defined therein by a device which includes at least one flexible tubular element defining a working channel therein, and an attachment structure configured to operatively engage the at least one flexible tubular element with the delivery device. The at least one flexible tubular element is shiftable between a collapsed delivery configuration with a reduced external profile over the delivery device, and an expanded working configuration with the working channel defined therein in an open configuration facilitating passage of an instrument therethrough.

In some embodiments, the at least one flexible tubular element has a closed distal end and is inflatable to shift the at least one flexible tubular element from the delivery configuration to the working configuration. In some embodiments, the device further includes an inflation port at a proximal end of the at least one flexible tubular element. In some embodiments, the at least one flexible tubular element has a closed distal end, and the device further including a stylet extending through the working channel of the at least one flexible tubular element and having a distal end configured to puncture the closed distal end of the at least one flexible tubular element to allow passage of an instrument therethrough.

In some embodiments, the device further includes a guide element extending through the working channel of the at least one flexible tubular element. In some embodiments, the guide element is configured to engage an instrument to guide the instrument through the working channel defined within the at least one flexible tubular element.

In some embodiments, the attachment structure is configured to snap over a distal end of the delivery device to couple the flexible tubular element with the delivery device. In some embodiments, the attachment structure is an o-ring.

In some embodiments, the device includes at least two flexible tubular elements.

In accordance with various principles of the present disclosure, a flexible tubular system is provided with a plurality of working channels configured for passage of a medical instrument therethrough, the system including a delivery device having a working channel defined therethrough; and at least one flexible tubular element configured to be coupled to the exterior of the delivery device and shiftable between a collapsed delivery configuration with a reduced external profile over the delivery device and an expanded working configuration with the working channel defined therein in an open configuration facilitating passage of an instrument therethrough.

In some embodiments, the flexible tubular system includes an attachment structure configured to couple the delivery device and the flexible tubular element. In some embodiments, the attachment structure is configured to snap over a distal end of the delivery device to couple the flexible tubular element thereto.

In some embodiments, the at least one flexible tubular element has a closed distal end and is inflatable to shift the at least one flexible tubular element from the delivery configuration to the working configuration. In some embodiments, the flexible tubular system further including a guide element extending through the working channel defined through the at least one flexible tubular element, and having a distal end configured to puncture the closed distal end of the at least one flexible tubular element to allow passage of an instrument therethrough.

In some embodiments, the flexible tubular system further including a guide element extending through the working channel of the at least one flexible tubular element.

In accordance with various principles of the present disclosure, a method of extending medical instruments to a treatment site within a patient's body includes attaching a collapsed flexible tubular element having a working channel defined therein to the exterior of a delivery device having a working channel defined therein; extending the distal ends of the flexible tubular element and the delivery device to the treatment site, and expanding the flexible tubular element to facilitate passage of a medical instrument therethrough.

In some embodiments, the method includes passing a first medical instrument through the working channel defined in the delivery device; and passing a second medical instrument through the working channel defined in the flexible tubular element.

In some embodiments, the method further includes guiding a medical instrument over a guide element within the flexible tubular element and to the treatment site. In some embodiments, the distal end of the flexible tubular element is closed, and the method further includes inflating the collapsed flexible tubular element to expand the flexible tubular element, puncturing the closed distal end of the flexible tubular element with the guide element, and extending the medical instrument through the punctured distal end of the flexible tubular element and to the treatment site.

In some embodiments, the method further includes inflating the collapsed flexible tubular element to expand the flexible tubular element.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an exploded perspective view of a flexible tubular system formed in accordance with various aspects of the present disclosure.

FIG. 2 illustrates a perspective view of a sheath system such as shown in FIG. 1 mounted on a delivery device as shown in FIG. 1, in a delivery configuration.

FIG. 3 illustrates a perspective view similar to that of FIG. 2, but in a working configuration.

FIG. 4 illustrates a perspective view similar to that of FIG. 3, but with closed ends of the delivery tubes of the sheath system opened.

FIG. 5 illustrates a perspective view similar to that of FIG. 4, but with instruments extending through the opened ends of the delivery tubes and the delivery device.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery) , and “distal” refers to the direction or location furthest from the user, such as when using the device (e.g., introducing the device into a patient, or during implantation, positioning, or delivery), and/or closest to a treatment site. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. It will be appreciated that a channel or bore as referenced herein need not have a circular cross-sectional shape.

Endoscopic Submucosal Dissection (ESD) is a minimally invasive, organ sparing technique for removal of cancerous and pre-cancerous lesions (>2 cm) from the gastrointestinal tract. To perform ESD, navigation to the lesion site is completed with an endoscope. Markings around the lesion are created using a monopolar ESD knife. A lifting solution may then be injected through an injection needle into the submucosa creating a separation of the lesion from the muscularis propria layer, and potentially reducing risk of perforation. Following this, an initial circumferential incision around the lesion is completed using a monopolar ESD knife. Most of the procedure is spent dissecting the submucosal plane; a layer comprised of loose connective tissue. As the region is resected, stability is reduced, and visualization becomes difficult.

Mastery of the ESD technique for the colorectal space (where the luminal walls are thinner and can be difficult to maneuver), the colon, and duodenum generally requires extensive training with an experienced endoscopist, and presents numerous challenges. Use of the ESD technique is generally considered technically challenging with a steep learning curve—especially in locations outside of gastric ESD. Thinner walls and difficulty maneuvering in the intestines make ESD complicated to perform. There is a risk of a variety of adverse events, such as perforation during endoscopic removal procedures as well as during delivery of instruments, tools, devices, etc., necessary for performing the procedure. The ESD procedure is considered relatively lengthy, with a mean ESD time for gastric/colonic resection calculated by some at approximately 115.8 minutes or even as long as approximately 176 minutes. Insufflation is currently required for ESD and can be painful for the patient. The devices used during ESD further present various challenges. The typically limited number of working channels available for passage of instruments, tools, devices to be used during the ESD procedure means multiple devices cannot be used at the same time to mimic laparoscopic GI surgery. There exists a continuing need for improved visualization. Furthermore, there exists a continuing need for improved stability (traction) of the lesion site, which may be difficult to achieve in view of at least the above-noted challenges.

In accordance with various principles of the present disclosure, a sheath system with one or more flexible tubular elements, and associated methods of use thereof provide a low-cost, optionally single use (e.g., disposable), solution for providing multiple working channels in a delivery device, such as an endoscope, used to deliver instruments, tools, devices (such terms being used interchangeably herein without intent to limit) to a treatment site during a medical/surgical procedure. The addition of working channels to a pre-existing device with limited working channels (e.g., a single working channel) allows additional devices to be used during procedures without impacting upon intubation capability. Also, the addition of working channels enables the user to create tension with one instrument, while cutting with the other.

Devices, systems, and methods formed in accordance with various principles of the present disclosure preferably do not functionally or operationally impact (e.g., significantly increase) the outer profile, such as the outer diameter, of the device (e.g., endoscope) to which the devices, systems, and methods are applied. For instance, navigation of the devices and systems within the body is not negatively impacted by addition of a device or system formed in accordance with various principles of the present disclosure. Moreover, devices and systems formed in accordance with various principles of the present disclosure add minimal additional bulk to the pre-existing device (e.g., endoscope) during navigation to a treatment site (e.g., intubation). For instance, the channels of devices and systems formed in accordance with various principles of the present disclosure may remain in a low-profile (e.g., collapsed, constrained, retracted, deflated, etc.) configuration until the treatment site has been reached. Once the devices/systems are at the treatment site, the channels may be inflated to create access for multiple additional devices to be delivered to the treatment site. An elongated device, such as a pre-loaded guide element, may be provided within the added working channel to guide a medical instrument therethrough, and/or to provide a pathway through which the medical instrument may access the treatment site at which the medical instrument is to be used.

In accordance with various aspects of the present disclosure, one or more flexible tubular elements are provided with an attachment structure sized, shaped, configured, and dimensioned to couple the one or more flexible tubular elements to a pre-existing tubular delivery device. It will be appreciated that reference is made herein generically to a delivery device as encompassing devices with working channels, such as endoscopes and the like, through which a medical tool, device, instrument, etc. may be moved. Movement of the medical devices through the working channel (and, for example, with respect to a treatment site) may be referenced herein as advanced, retracted, passed, extended, navigated, transported, conveyed, manipulated, etc., including other grammatical forms thereof, without intent to limit.

Various embodiments of a device, system, and method for providing additional working channels to a delivery device will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein, and the examples of embodiments disclosed herein are not intended as limiting the broader aspects of the present disclosure.

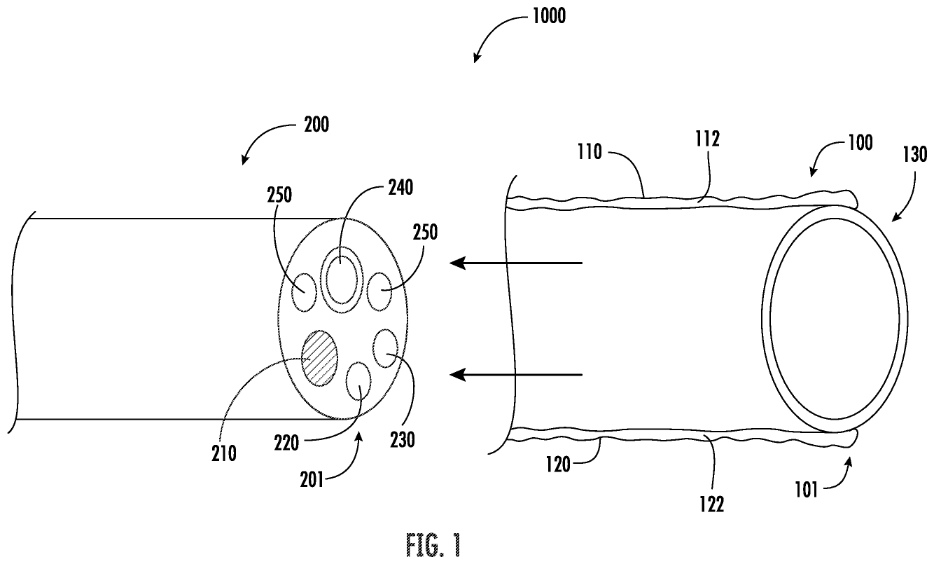

Turning now to the drawings, an example of an embodiment of a device 100 providing additional working channels to a delivery device 200 having a limited number of working channels defined therein is illustrated in FIG. 1. The illustrated delivery device 200 is in the form of a flexible tubular element with at least one working channel 210 extending longitudinally therethrough. The device 100 providing the additional working channels is referenced herein as a sleeve or sheath system 100 for the sake of convenience and without intent to limit. Although the sheath system 100 is illustrated as being configured to add to the delivery device 200 two additional flexible tubular elements 110, 120 defining working channels 112, 114 therein, the sheath system 100 may add only one, or more than two working channels to the delivery device 200.

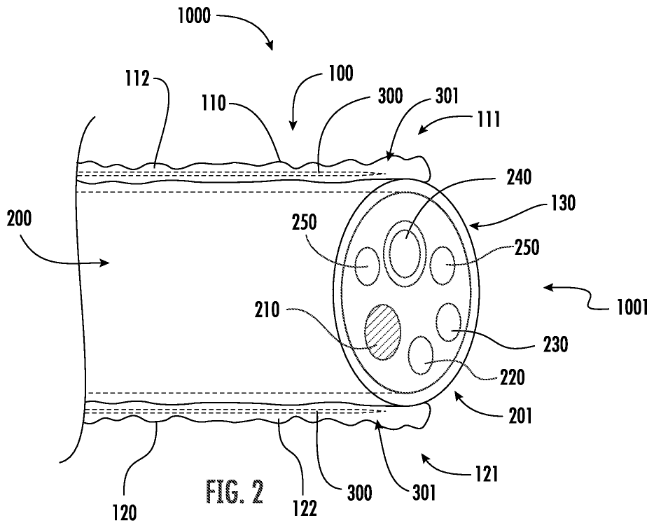

As illustrated in FIG. 1, the sheath system 100 may include an attachment structure 130 configured to facilitate engagement of the sheath system 100 with the delivery device 200. For instance, the attachment structure 130 may be configured to mount the sheath system 100 with respect to (over, along, etc.) the delivery device 200. In the example of an embodiment of a sheath system 100 illustrated in FIG. 1, the attachment structure 130 is provided at the distal end 101 of the sheath system 100 to engage with the distal end 201 of the delivery device 200. The delivery device 200 may include a plurality of channels, including, without limitation, working channel 210 (such as for passage of instruments therethrough), an aspiration channel 220 (such as for an air nozzle), an irrigation channel 230 (such as for a water nozzle or water jet), an imaging channel 240 (e.g., for a camera, an associated objective lens, etc.), and a light guide channel 250 (more than one may be beneficial for a light, such as an optic fiber, for illumination purposes for the imaging device). The attachment structure 130 may be placed a sufficient distance proximal to the distal end 201 of the delivery device 200 so as not to interfere with the open end of any of the channels 210, 220, 230, 240, 250 opening at the distal end 201 of the delivery device 200, or operations performed therethrough (e.g., aspiration, irrigation, visualization, etc.) or by instruments extending therethrough. The attachment structure 130 preferably is configured to securely engage the sheath system 100 with respect to the delivery device 200 to remain in place (e.g., not slide or otherwise move distally or proximally, such as with respect to the delivery device 200) while being navigated through potentially tortuous internal body passages, such as illustrated in FIG. 2. For instance, the attachment structure 130 may be sized, shaped, configured, and dimensioned to snap over the distal end 201 of the delivery device 200 to couple the sheath system 100 thereto, such as to grip the exterior of the delivery device 200. In some embodiments, the attachment structure 130 may be an o-ring, which may be expanded, such as with the use of a tool (e.g., pliers, circlip pliers, etc.), to be inserted over the distal end 201 of the delivery device 200. It will be appreciated that other configurations and arrangements and positions of an attachment structure are within the scope and spirit of the present disclosure.

Once the distal end 101 of the sheath system 100 is engaged with the distal end 201 of the delivery device 200, the additional flexible tubular elements 110, 120 are extended or slid proximally over the delivery device 200 to a proximal end of the delivery device 200. In some embodiments, the proximal end of the sheath system 100 operatively engages a handle at the proximal end of the delivery device 200. It will be appreciated that the proximal ends of the sheath system 100 and the delivery device 200, and the handle, may be in any form known to those of ordinary skill in the art and are not limited by the present disclosure and thus, for the sake of simplicity, are not illustrated. In some embodiments, such as discussed in greater detail below, a hub and/or inflation ports are provided at the proximal end of one or both of the flexible tubular elements 110, 120 of the sheath system 100 (not shown, but in a form as known to those of ordinary skill in the art and not limited by the present disclosure) for coupling with an inflation device. The flexible tubular elements 110, 120 of the sheath system 100 may be extended proximally and coupled to a proximal end of the delivery device 200 so that the proximal ends of the flexible tubular elements 110, 120 are taut.

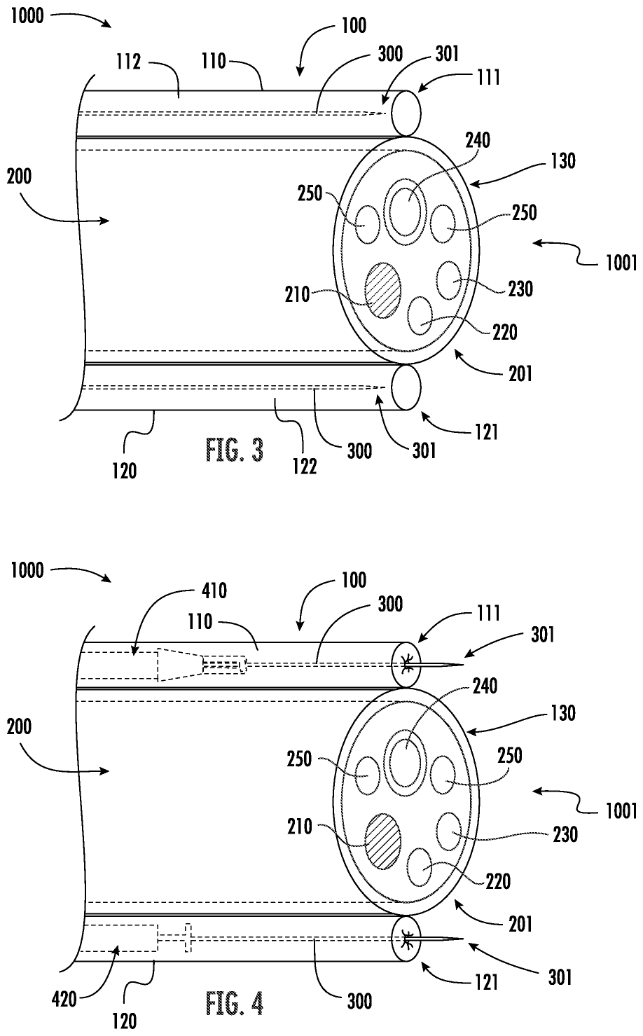

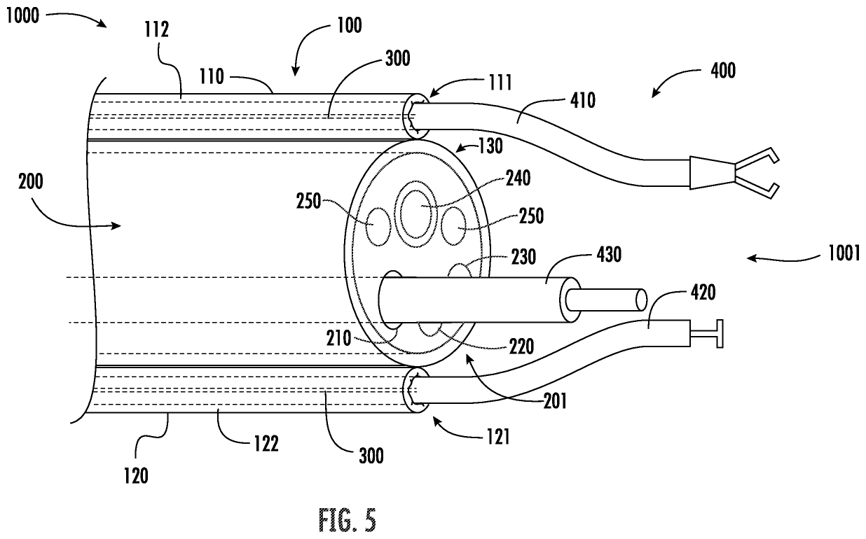

The flexible tubular elements 110, 120 of the sheath system 100 may be formed of a thin flexible material capable of shifting the working channels 112, 122 therein between a delivery configuration, such as illustrated in FIG. 2, and a working configuration, such as illustrated in FIG. 3, FIG. 4, and FIG. 5. In the delivery configuration, the flexible tubular elements 110, 120 are sized, shaped, configured, and dimensioned to minimize the space occupied by the flexible tubular elements 110, 120 along the delivery device 200, and the passages therethrough are generally at a reduced size not optimal for passing instruments therethrough. It will be appreciated that terms such as reduced, unexpanded, deflated, compact, contracted, collapsed, etc., and other grammatical forms thereof, may be used interchangeably herein and without intent to limit. Such terms may be used herein to describe the smaller configuration of the flexible tubular elements 110, 120 and/or the working channels 112, 122 when in a delivery configuration in which the flexible tubular elements 110, 120 add minimal bulk to the delivery device 200. Overall, the flexible tubular system 1000 made up of the sheath system 100 and the delivery device 200, such as illustrated in FIG. 2, does not have a cross-sectional area or overall size significantly larger than that of the delivery device 200 alone (without the sheath system 100), such that the sheath system 100 does not increase bulk around the circumference of the delivery device 200 during navigation of the flexible tubular system 1000 through the body. In contrast, in a working configuration, the working channels 112, 122 through the flexible tubular elements 110, 120 are sized, shaped, configured, and dimensioned to facilitate passage of instruments therethrough. For instance, in the working configuration the flexible tubular elements 110, 120 and/or the passages therethrough are increased, expanded, inflated, enlarged, etc., (such terms being used interchangeably herein without intent to limit) relative to the reduced delivery configuration, such as in a manner described in further detail below. The material of the flexible tubular elements 110, 120 preferably is also sufficiently strong to withstand any forces imparted by instruments passing therethrough. In some embodiments, the material may be reinforced, such as with braiding or other techniques known to those of ordinary skill in the art and not limiting the present disclosure. A lubricious material may also be beneficial to facilitate passing of the flexible tubular elements 110, 120 through potentially tortuous and/or narrow body passages. Moreover, a lubricious material may facilitate passage of instruments through the working channels 112, 122, such as by reducing the likelihood of the instruments stopping, catching, getting caught up, etc., during navigation, which may perforate the flexible tubular element 110, 120 and potentially may also puncture/perforate the body passage through which the flexible tubular system 1000 passes. Examples of suitable materials include biocompatible materials such as but not limited to polytetrafluorethylene (PTFE), nylon, polyamide, polyetheretherketone (PEEK) and Pebax® elastomers (polyether block amide thermoplastic elastomers sold by Arkema). The material should be flexible with wall thicknesses of approximately 0.5 mm±0.05 mm depending upon material selection and fabrication method.

In accordance with various principles of the present disclosure, the sheath system 100 is inserted over the delivery device 200 with the additional flexible tubular elements 110, 120 in a reduced delivery configuration, such as illustrated in FIG. 1, and FIG. 2. Generally, devices which increase the outer diameter of an endoscope during intubation make navigation of internal body passages such as the colon much more difficult. A sheath system 100 formed in accordance with various principles of the present disclosure advantageously has a delivery configuration facilitating navigation and delivery through potentially tortuous and/or narrow internal body passages. The flexible tubular elements 110, 120 would remain in a deflated configuration so as not to increase bulk around the circumference of the delivery device 200 during intubation. For instance, the sheath system 100 may be configured to conform to the outer diameter of the delivery device 200.

Once intubation is complete and the distal end 1001 of the flexible tubular system 1000, made up of the sheath system 100 and the delivery device 200, reaches a treatment site, the flexible tubular elements 110, 120 may be shifted into a working configuration, such as illustrated in FIG. 3. In the working configuration, the working channels 112, 122 through the flexible tubular elements 110, 120 are expanded from the delivery configuration to a working configuration to create access therein for devices to be conveyed therethrough, through the sheath system 100 and to the treatment site. In accordance with various principles of the present disclosure, the flexible tubular elements 110, 120 of the sheath system 100 may be inflated to shift from a collapsed delivery configuration to an expanded working configuration. Optionally, though not necessarily, the material of the flexible tubular elements 110, 120 may be somewhat elastic, and/or reinforced to allow expansion without bursting. The respective distal ends 111, 121 of the flexible tubular elements 110, 120 would be closed (e.g., formed as closed ends) or sealed initially to assist with inflation once at the desired site (e.g., a treatment site). Air or other inflation medium may be supplied at the proximal ends of the flexible tubular elements 110, 120 (e.g., via ports such as described above) to inflate the flexible tubular elements 110, 120 into a working configuration through which instruments may be readily navigated. It will be appreciated that the size, shape, configuration, and dimensions of the flexible tubular elements 110, 120 and the working channels 112, 122 therein may be selected based on the corresponding size, shape, configuration, and dimensions of the instruments to be navigated therethrough.

In accordance with various principles of the present disclosure, at least one of the working channels 112, 122 may be pre-loaded with a guidewire or stylet 300 (hereinafter, reference is made simply to a stylet for the sake of convenience and without intent to limit), as illustrated in FIG. 3. The stylet 300 may be a solid flexible elongated member, with a diameter of approximately at least about 0.01″ (0.254 mm) and at most about 0.02″ (0.0308 mm). If the stylet 300 is in the form of a guidewire, it may have a metal core with a plastic outer coating or covering. The stylet 300 may be a guide element facilitating guiding of the sheath system 100 through a body passage. Additionally or alternatively, the stylet 300 may be a guide element over which a device may be tracked through the working channels 112, 122. The tools, instruments, devices, etc., (hereinafter instruments 400, generally, for the sake of simplicity and without intent to limit) which are passed through the working channels 112, 122, as illustrated in FIG. 4, optionally have through holes or channels through which the stylet 300 may pass so that the instruments 400 may ride over and along the stylet 300, thereby to be guided by the stylet 300 to the treatment site.

Optionally, the stylets 300 may act as a puncture mechanism once instruments 400 have been tracked to the distal ends 111, 121 of the flexible tubular elements 110, 120. The stylets 300 may be formed with a distal end 301 configured to puncture the closed distal ends 111, 121 of the flexible tubular elements 110, 120, such as illustrated in FIG. 4. If the stylet 300 is a guidewire, the distal end 301 may be an exposed steel end shaped (e.g., sufficiently sharp) to puncture the closed distal ends 111, 121 of the flexible tubular elements 110, 120. The instruments 400 within the working channels 112, 122 of the sheath system 100 may then be advanced distally to the treatment site to be used for the procedure to be performed at the treatment site, such as illustrated in FIG. 5. The instruments 400 may be any known or heretofore known instruments in the art, the present disclosure not being limited by the forms or functions thereof. Although various instruments 410, 420, 430 are shown advanced through respective working channels 112, 122, 210, it will be appreciated that an instrument need not extend through each working channels 112, 122, 210, or may be extended at different times rather than simultaneously as shown. It will be appreciate that once the distal ends 111, 121 of inflated flexible tubular elements 110, 120 have been punctured or otherwise disrupted, the flexible tubular elements 110, 120 may deflate.

Devices and systems as described above, and methods of use thereof, provide a variety of benefits over currently available devices and systems for delivering medical tools to a treatment site. For instance, procedural times are shortened by the availability of more than one working channel via which a tool can be delivered to the treatment site. Learning curves for a procedure, such as ESD, may thereby be lowered. Various aspects of the present disclosure reduce rates of adverse events such as perforation and delayed bleeding. Moreover, various aspects of the present disclosure facilitate performance of various medical procedures. For instance, the above and other benefits generally lead to improved en bloc resection of lesions in the gastrointestinal tract, such as in ESD procedures. The various principles of the present disclosure enable a tension method which allows manipulation of tissue in a small space, such as permitted by Boston Scientific Corporation's Orise© procedure utilizing injection of a specialized submucosal lifting agent into tissue to facilitate performance of various procedures (e.g., resection) on the tissue. It will be appreciated that principles of the present disclosure may be applied to other procedures, such as other endoscopic procedures, such as endoscopic retrograde cholangiopancreatography (ERCP), endoscopic ultrasound (EUS), etc. Various additional benefits such as cost reduction, improved patient safety, etc., may also be realized by various aspects of the present disclosure.

Various modifications to the above-described example of an embodiment of a sheath system are within the scope of the present disclosure. For instance, a sheath system formed in accordance with various principles of the present disclosure may be further enhanced by adding three or four or five or six or more working channels to the delivery device to allow even more therapy for various (e.g., endoscopic) procedures. The tubular elements defining the additional working channels may also potentially aid with insufflation and stability of the delivery device.

It will be appreciated that a delivery device to be used with the present disclosure is not limited by the illustrated and described embodiment, and may include a variety of medical devices for accessing body passageways, including, for example, duodenoscopes, catheters, ureteroscopes, bronchoscopes, colonoscopes, arthroscopes, cystoscopes, hysteroscopes, and the like.

The medical devices, instruments, tools, etc., to be navigated through the working channels of the tubular system of the present disclosure are not limited, and may include a variety of medical devices, instruments, tools, etc. For instance, the devices, instruments, tools, etc., may be used to perform a procedure or operation which is either diagnostic or therapeutic or both, such as grasping, lifting, resecting, dissecting, retracting, cutting, suturing, stapling, injecting, cauterizing, clipping, and/or otherwise manipulating tissue. Such devices, instruments, tools, etc., include graspers (e.g., a rotatable grasping clip, such as a RESOLUTION™ clip device sold by Boston Scientific Corporation, with a pair of jaws/arms, etc.), cutting tool (e.g., knife, electrocautery device, scissors), snares, etc.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A device for providing an additional external working channel with respect to a delivery device with at least one working channel defined therein, said device comprising:
at least one flexible tubular element defining a working channel therein; and
an attachment structure configured to operatively engage said at least one flexible tubular element with said delivery device;
wherein said at least one flexible tubular element is shiftable between a collapsed delivery configuration with a reduced external profile over the delivery device, and an expanded working configuration with the working channel defined therein in an open configuration facilitating passage of an instrument therethrough.

2. The device of claim 1, wherein said at least one flexible tubular element has a closed distal end and is inflatable to shift said at least one flexible tubular element from the delivery configuration to the working configuration.

3. The device of claim 2, further comprising an inflation port at a proximal end of said at least one flexible tubular element.

4. The device of claim 2, wherein said at least one flexible tubular element has a closed distal end, said device further comprising a stylet extending through the working channel of said at least one flexible tubular element and having a distal end configured to puncture the closed distal end of said at least one flexible tubular element to allow passage of an instrument therethrough.

5. The device of claim 1, further comprising a guide element extending through the working channel of said at least one flexible tubular element.

6. The device of claim 5, wherein said guide element is configured to engage an instrument to guide the instrument through the working channel defined within said at least one flexible tubular element.

7. The device of claim 1, wherein said attachment structure is configured to snap over a distal end of the delivery device to couple said flexible tubular element with said delivery device.

8. The device of claim 7, wherein said attachment structure is an o-ring.

9. The device of claim 1, wherein said device comprises at least two flexible tubular elements.

10. A flexible tubular system having a plurality of working channels configured for passage of a medical instrument therethrough, said flexible tubular system comprising:
a delivery device having a working channel defined therethrough; and
at least one flexible tubular element configured to be coupled to the exterior of said delivery device and shiftable between a collapsed delivery configuration with a reduced external profile over said delivery device and an expanded working configuration with the working channel defined therein in an open configuration facilitating passage of an instrument therethrough.

11. The flexible tubular system of claim 10, further comprising an attachment structure configured to couple said delivery device and said flexible tubular element.

12. The flexible tubular system of claim 11, wherein said attachment structure is configured to snap over a distal end of said delivery device to couple said flexible tubular element thereto.

13. The flexible tubular system of claim 10, wherein said at least one flexible tubular element has a closed distal end and is inflatable to shift said at least one flexible tubular element from the delivery configuration to the working configuration.

14. The flexible tubular system of claim 13, further comprising a guide element extending through the working channel defined through said at least one flexible tubular element, and having a distal end configured to puncture the closed distal end of said at least one flexible tubular element to allow passage of an instrument therethrough.

15. The flexible tubular system of claim 10, further comprising a guide element extending through the working channel of said at least one flexible tubular element.

16. A method of extending medical instruments to a treatment site within a patient's body, said method comprising:
attaching a collapsed flexible tubular element having a working channel defined therein to the exterior of a delivery device having a working channel defined therein;
extending the distal ends of the flexible tubular element and the delivery device to the treatment site; and
expanding the flexible tubular element to facilitate passage of a medical instrument therethrough.

17. The method of claim 16, further comprising:
passing a first medical instrument through the working channel defined in the delivery device; and
passing a second medical instrument through the working channel defined in the flexible tubular element.

18. The method of claim 16, further comprising guiding a medical instrument over a guide element within the flexible tubular element and to the treatment site.

19. The method of claim 18, wherein the distal end of the flexible tubular element is closed, said method further comprising:
inflating the collapsed flexible tubular element to expand the flexible tubular element;
puncturing the closed distal end of the flexible tubular element with the guide element; and
extending the medical instrument through the punctured distal end of the flexible tubular element and to the treatment site.

20. The method of claim 16, further comprising inflating the collapsed flexible tubular element to expand the flexible tubular element.
